(19) United States
(12) Patent Application Publication
(10) Pub. No.: US 2006/0128632 A1
(43) Pub. Date: Jun. 15, 2006

(54) Peptide inhibitors of smac protein binding to inhibitor of apoptosis proteins (iap)

(76) Inventors: Sushil Kumil Sharma, West Orange, NJ (US); Leigh Zawel, Hingham, MA (US); Mark G. Palermo, Rindge, NH (US); Nagarajan Chandeamouli, Morristown, NJ (US); Kenneth Walter Bair, Oakland, CA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/519,042

(22) PCT Filed: Jul. 1, 2003
(86) PCT No.: PCT/EP03/07005

§ 371 Date: Jul. 13, 2005

Related U.S. Application Data
(60) Provisional application No. 60/393,150, filed on Jul. 2, 2002.

(51) Int. Cl. A61K 38/05; A61K 38/04; C07K 5/06; C07K 5/04; C07C 237/18

(52) U.S. Cl. 514/18; 514/19; 548/532; 530/331; 564/152

(57) ABSTRACT

The present disclosure relates to XIAP inhibitor compounds of the formula I 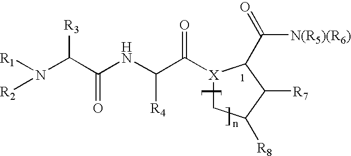 wherein the substituents are as described in the specification. The inventive compounds are useful as therapeutic agents for the treatment of proliferative disorders, including cancer.

The present invention relates generally to novel compounds that inhibit the binding of the Smac protein to inhibitor of Apoptosis Proteins (IAP). The present invention includes novel compounds, novel compositions, methods of their use and methods of their manufacture, where such compounds are generally pharmacologically useful as agents in therapies whose mechanism of action rely on the inhibition of the Smac/IAP interaction, and more particularily useful in therapies for the treatment of proliferative diseases, including cancer.

Programmed cell death plays a critical role in regulating cell number and in eliminating stressed or damaged cells from normal tissues. Indeed, the network of apoptotc signalling mechanisms inherent in most cell types provides a major barrier to the development and progression of human cancer. Since most commonly used radiation and chemo-therapies rely on activation of apoptotic pathways to kill cancer cells, tumor cells which are capable of evading programmed cell death often become resistant to treatment.

Apoptosis signalling networks are classified as either intrinsic when mediated by death receptor-ligand interactions or extrinsic when mediated by cellular stress and mitochondrial permeabilization. Both pathways ultimately converge on individual Caspases. Once activated, Caspases cleave a number of cell death-related substrates, effecting destruction of the cell.

Tumor cells have devised a number of strategies to circumvent apoptosis. One recently reported molecular mechanism involves the overexpression of members of the IAP family. IAPs sabotage apoptosis by directly interacting with and neutralizing Caspases. The prototype IAP, XIAP, has three functional domains referred to as BIR 1, 2 & 3 domains. BIR3 interacts directly with Caspase 9 and inhibits its ability to bind and cleave its natural substrate, Procaspase 3.

It has been reported that a proapoptotic mitochondrial protein, Smac (also known as DIABLO), is capable of neutralizing XIAP by binding to a peptide binding pocket (Smac binding site) on the surface of BIR3 thereby precluding interaction between XIAP and Caspase 9. The present invention relates to therapeutic molecules that bind to the Smac binding pocket thereby promoting apoptosis in rapidly dividing cells. Such therapeutic molecules are useful for the treatment of proliferative diseases, including cancer.

The present invention relates to compounds of the formula (I) 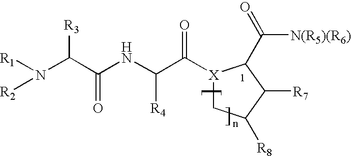
wherein

R₁ is H;

R₂ is H, C₁-C₄alkyl which is unsubstituted or substituted by one or more substituents selected from halogen, —OH, —SH, —OCH₃, —SCH₃, —CN, —SCN and nitro;

R₃ is H, —CF₃, —C₂F₅, —CH₂-Z or R₂ and R₃ together form with the nitrogen form a C₃-C₆heteroaliphatic ring;

Z is H, —OH, F, Cl, —CH₃; —CF₃, —CH₂Cl, —CH₂F or —CH₂OH;

R₄ is C₁-C₁₆ straight chain alkyl, C₃-C₁₀ branched chain alkyl, —(CH₂)₀₋₆—C₃-C₇cycloalkyl, —(CH₂)₁₋₆Z₁, —(CH₂)₀₋₆-phenyl, and —(CH₂)₀₋₆-het, wherein the alkyl, cycloalkyl and phenyl substituents are unsubstituted or substituted;

Z₁ is —N(R₉)—C(O)—C₁-C₁₀alkyl, —N(R₉)—C(O)—(CH₂)₁₋₆—C₃-C₇-cycloalkyl, —N(R₉)—C(O)—(CH₂)₀₋₆-phenyl, —N(R₉)—C(O)—(CH₂)₁₋₆-het, —C(O)—N(R₁₀)(R₁₁), —C(O)—O—C₁-C₁₀alkyl, —C(O)—O—(CH₂)₁₋₆—C₃-C₇-cycloalkyl, —C(O)—O—(CH₂)₀₋₆-phenyl, —C(O)—O—(CH₂)₁₋₆het, —O—C(O)—C₁-C₁₀alkyl, —O—C(O)—(CH₂)₁₋₆—C₃-C₇-cycloalkyl, —O—C(O)—(CH₂)₀₋₆-phenyl, —O—C(O)—(CH₂)₁₋₆-het, wherein the alkyl, cycloalkyl and phenyl substituents are unsubstituted or substituted;

het is a 5-7 membered heterocyclic ring containing 1, 2 or 3 heteroatoms selected from N, O and S, or an 8-12 membered fused ring system including at least one 5-7 membered heterocyclic ring containing 1, 2 or 3 heteroatoms selected from N, O, and S, which heterocyclic ring or fused ring system is unsubstituted or substituted on a carbon atom by halogen, hydroxy, C₁-C₄alkyl, C₁-C₄ alkoxy, nitro, —O—C(O)—C₁-C₄alkyl or —C(O)—O—C₁-C₄-alkyl or on a nitrogen by C₁-C₄ alkyl, —O—C(O)—C₁-C₄alkyl or —C(O)—O—C₁-C₄-alkyl;

R₉ is H, —CH₃, —CF₃, —CH₂OH or CH₂Cl;

R₁₀ and R₁₁ are each independently H, C₁-C₄alkyl, C₃-C₇-cycloalkyl, —(CH₂)₁₋₆—C₃-C₇-cycloalkyl, —(CH₂)₀₋₆-phenyl, wherein the alkyl, cycloalkyl and phenyl substituents are unsubstituted or substituted, or R₁₀ and R₁₁ together with the nitrogen are het;

X is CH or N;

R₅ is H, C₁-C₁₀-alkyl, C₃-C₇-cycloalkyl, —(CH₂)₁₋₆—C₃-C₇-cycloalkyl, —C₁-C₁₀-alkyl-aryl, —(CH₂)₀₋₆—C₃-C₇-cycloalkyl-(CH₂)₀₋₆-phenyl, —(CH₂)₀₋₄CH—((CH₂)₁₋₄-phenyl)₂, —(CH₂)₀₋₆—CH(phenyl)₂, —C(O)—C₁-C₁₀alkyl, —C(O)—(CH₂)₁₋₆—C₃-C₇-cycloalkyl, —C(O)—(CH₂)₀₋₆-phenyl, —(CH₂)₁₋₆-het, —C(O)—(CH₂)₁₋₆-het, or R₅ is a residue of an amino acid, wherein the alkyl, cycloalkyl, phenyl and aryl substituents are unsubstituted or substituted;

R₆ is H, methyl, ethyl, —CF₃, —CH₂OH or —CH₂Cl; or

R₅ and R₆ together with the nitrogen are het;

R₇ and R₈ are cis relative to the acyl substituent at the one position of the ring and are each independently H, —C₁-C₁₀ alkyl, —OH, —O—C₁-C₁₀-alkyl, —(CH₂)₀₋₆—C₃-C₇-cycloalkyl, —O—(CH₂)₀₋₆-aryl, phenyl, —(CH₂)₁₋₆-het, —O—(CH₂)₁₋₆-het, —N(R₁₂)(R₁₃), —S—R₁₂, —S(O)—R₁₂, —S(O)₂—R₁₂, —S(O)₂—NR₁₂R₁₃ wherein the alkyl, cycloalkyl and aryl substituents are unsubstituted or substituted;

R₁₂ and R₁₃ are independently H, C₁-C₁₀ alkyl, —(CH₂)₀₋₆—C₃-C₇-cycloalkyl, —(CH₂)₀₋₆—(CH)₀₋₁(aryl)₁₋₂, —C(O)—C₁-C₁₀alkyl, —C(O)—(CH₂)₁₋₆—C₃-C₇-cycloalkyl, —C(O)—O—(CH₂)₀₋₆-aryl, —C(O)—(CH₂)₀₋₆—O-fluorenyl, —C(O)—NH—(CH₂)₀₋₆-aryl, —C(O)—(CH₂)₀₋₆-aryl, —C(O)—(CH₂)₁₋₆-het, wherein the alkyl, cycloalkyl and aryl substituents are unsubstituted or substituted; or a substituent that facilitates transport of the molecule across a cell membrane, or R₁₂ and R₁₃ together with the nitrogen are het;

aryl is phenyl or naphthyl which is unsubstituted or substituted;

n is 0, 1 or 2;

and wherein

substituted alkyl substituents are substituted by one or more substituents selected from a double bond, halogen, OH, —O—C₁-C₆alkyl, —S—C₁-C₆alkyl and —CF₃; substituted cycloalkyl substituents are substituted by one or more substituents selected from a double bond, C₁-C₆alkyl, halogen, OH, —O—C₁-C₆alkyl, —S—C₁-C₆alkyl and —CF₃; and substituted phenyl or aryl are substituted by one or more substituents selected from halogen, hydroxy, C₁-C₄ alkyl, C₁-C₄ alkoxy, nitro, —CN, —O—C(O)—C₁-C₄alkyl and —C(O)—O—C₁-C₄-alkyl.

Unsubstituted is intended to mean that hydrogen is the only substituent.

Halogen is fluorine, chlorine, bromine or iodine, especially fluorine and chlorine.

Unless otherwise specified alkyl substituents include straight or branched chain alkyl, such as methyl, ethyl, n-propyl, isopropyl, n-butyl, sec-butyl, tert-butyl, n-pentyl and branched pentyl, n-hexyl and branched hexyl, and the like.

Cycloalkyl substituents include cyclopropyl, cyclobutyl, cyclopentyl, cyclohexyl, and cycloheptyl.

In a particularly important embodiment of the present invention, R₃ and R₄ have the stereochemistry indicated in formula II, with the definitions of the variable substituents and preferences described herein also applying to compounds having the stereochemistry indicated in formula II. 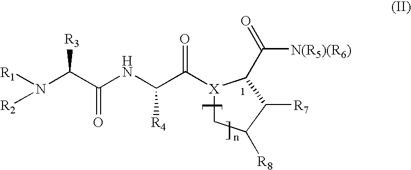
R₂ is especially H, methyl or ethyl, particularly H or methyl, which methyl group is unsubstituted or substituted, particularly unsubstituted methyl. R₂ as substituted methyl especially includes chloromethyl, dichloromethyl and especially trifluoromethyl.

R₃ is especially methyl.

In a particular embodiment, R₂ and R₃ together with the nitrogen form a heteroaliphatic ring, including saturated and unsaturated 3 to 6 membered nonaromatic rings, for example, aziridine, azetidine, azole, piperidine, piperazine, and the like, especially aziridine and azetidine.

R₄ is especially C₁-C₄alkyl or C₃-C₇ cycloalkyl particularly isopropyl or cyclohexyl.

R₅ as —(CH₂)₀₋₆—C₃-C₇-cycloalkyl-(CH₂)₀₋₆-phenyl includes fused cycloalkyl-phenyl rings, such as indanyl, when there are no methylenes between the cycloalkyl and phenyl rings.

R₅ as —(CH₂)₀₋₄CH—((CH₂)₁₋₄-phenyl)₂ is especially —CH(CH₂-phenyl)₂

R₆ is especially H.

A particularly important embodiment includes the compounds wherein R₅ is —C₁-C₄-alkyl-phenyl, especially those wherein R₅ is —C₂H₄-phenyl and R₆ is H.

In a particular embodiment, n is preferably 1.

In a particular embodiment of the present invention, one or both of R₇ and R₈ is H. If one of R₇ and R₈ is other than H, it is especially hydroxy, —N(R₁₂)(R₁₃), especially wherein R₁₂ is —C(O)—(CH₂)₁₋₆—C₃-C₇-cycloalkyl, for example, wherein (CH₂)₁₋₆—C₃-C₇-cycloalkyl is cyclohexylmethyl, —O—(CH₂)₀₋₆-aryl, for example, wherein (CH₂)₀₋₆-aryl is benzyl. If only one of R₇ and R₈ is other than H, it is preferred for R₈ to be the substituent other than H.

In a preferred embodiment, R₆ is H and R₅ is —C₁-C₁₀-alkyl-aryl, particularly phenylmethyl, phenylethyl and phenylpropyl, especially phenylethyl.

The het substituents include aromatic and non-aromatic heterocyclic rings and fused rings containing aromatic and non-aromatic heterocyclic rings. Suitable het substituents include unsubstituted and substituted pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, 1,4-oxathiapane, furyl, thienyl, pyrrole, pyrazole, triazole, thiazole, oxazole, pyridine, pyrimidine, isoxazolyl, pyrazine, quinoline, isoquinoline, pyridopyrazine, pyrrolopyridine, furopyridine, indole, benzofuran, benzothiofuran, benzindole, benzoxazole, pyrroloquinoline, and the like. The het substituents are unsubstituted or substituted on a carbon atom by halogen, especially fluorine or chlorine, hydroxy, C₁-C₄ alkyl, such as methyl and ethyl, C₁-C₄ alkoxy, especially methoxy and ethoxy, nitro, —O—C(O)—C₁-C₄alkyl or —C(O)—O—C₁-C₄-alkyl or on a nitrogen by C₁-C₄ alkyl, especially methyl or ethyl, —O—C(O)—C₁-C₄alkyl or —C(O)—O—C₁-C₄-alkyl, such as carbomethoxy or carboethoxy.

When two substituents together with a commonly bound nitrogen are het, it is understood that the resulting heterocyclic ring is a nitrogen-containing ring, such as aziridine, azetidine, azole, piperidine, piperazine, morphiline, pyrrole, pyrazole, thiazole, oxazole, pyridine, pyrimidine, isoxazolyl, and the like.

The amino acid residues include a residue of a standard amino acid, such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine. The amino acid residues also include the side chains of uncommon and modified amino acids. Uncommon and modified amino acids are known to those of skill in the art (see for example G. B. Fields, Z. Tiam and G Barany; Synthetic Peptides A Users Guide, University of Wisconsin Biochemistry Center, Chapter 3, (1992)) and include amino acids such as 4-hydroxyproline, 5-hydroxylysine, desmosine, beta-alanine, alpha, gamma- and beta-aminobutric acid, homocysteine, homoserine, citrulline, ornithine, 2- or 3-amino adipic acid, 6-aminocaproic acid, 2- or 3-aminoisobutric acid, 2,3-diaminopropionic acid, diphenylalanine, hydroxyproline and the like. If the side chain of the amino acid residue contains a derivatizable group, such as COOH, —OH or amino, the side chain may be derivatized by a substituent that reacts with the derivatizable group. For example, acidic amino acids, like aspartic and glutamic acid, or hydroxy substituted side chains, like those of serine or threonine, may be derivatized to form an ester, or amino side chains may form amide or alkylamino derivatives. In particular, the derivative may be a substituent that facilitates transport across a cell membrane. In addition, any carboxylic acid group in the amino acid residue, for example, an alpha carboxylic acid group, may be derivatized as discussed above to form an ester or amide.

Substituents that facilitate transport of the molecule across a cell membrane are known to those of skill in the medicinal chemistry arts (see, for example, Gangewar S., Pauletti G. M., Wang B., Siahaan T. J., Stella V. J., Borchardt R. T., Drug Discovery Today, vol. 2. p 148-155 (1997) and Bundgaard H. and Moss J., Pharmaceutical Research, vol. 7, p 885 (1990)). Generally, such substituents are lipophillic substituents. Such lipophillic substituents include a C₆-C₃₀ alkyl which is saturated, monounsaturated, polyunsaturated, including methylene-interrupted polyene, phenyl, phenyl which substituted by one or two C₁-C₈ alkyl groups, C₅-C₉ cycloalkyl, C₅-C₉ cycloalkyl which is substituted by one or two C₁-C₈ alkyl groups, —X₁-phenyl, —X₁-phenyl which is substituted in the phenyl ring by one or two C₁-C₈ alkyl groups, X₁—C₅-C₉ cycloalkyl or X₁—C₅-C₉ cycloalkyl which is substituted by one or two C₁-C₈ alkyl groups; where X₁ is C₁-C₂₄ alkyl which is saturated, monounsaturated or polyunsaturated and straight or branched chain.

It will be apparent to one of skill in the art when a compound of the invention can exist as a salt form, especially as an acid addition salt or a base addition salt. When a compound can exist in a salt form, such salt forms are included within the scope of the invention. Although any salt form may be useful in chemical manipulations, such as purification procedures, only pharmaceutically acceptable salts are useful for pharmaceutically products.

Pharmaceutically acceptable salts include, when appropriate, pharmaceutically acceptable base addition salts and acid addition salts, for example, metal salts, such as alkali and alkaline earth metal salts, ammonium salts, organic amine addition salts, and amino acid addition salts, and sulfonate salts. Acid addition salts include inorganic acid addition salts such as hydrochloride, sulfate and phosphate, and organic acid addition salts such as alkyl sulfonate, arylsulfonate, acetate, maleate, fumarate, tartrate, citrate and lactate. Examples of metal salts are alkali metal salts, such as lithium salt, sodium salt and potassium salt, alkaline earth metal salts such as magnesium salt and calcium salt, aluminum salt, and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

The compounds of formula (I) may be prepared as depicted below in scheme 1:

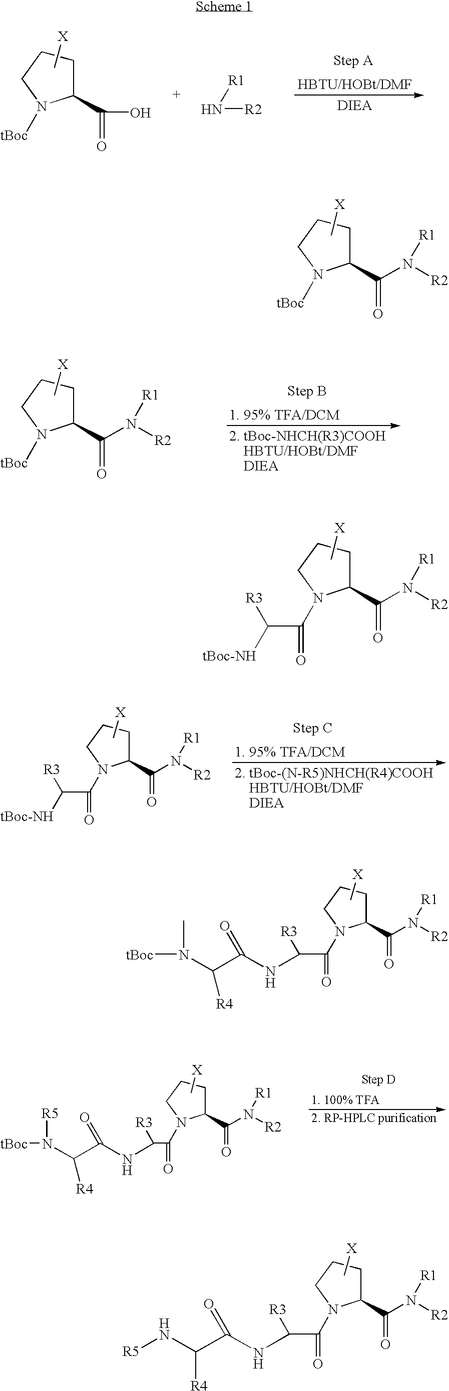

Step A: This step involves the coupling of an amine with t-Boc-L-Proline or its derivative with an amine using standard peptide coupling agents such as DIC/HOBt or HBTU/HOBt.

Step B: This step involves the removal of t-Boc group with trifluoroacetic acid (TFA) followed by coupling with a Boc protected natural or unnatural amino acid using standard peptide coupling agent.

Step C: This step involves the removal of t-Boc group with trifluoroacetic acid (TFA) followed by coupling with a Boc protected natural or unnatural amino acid using standard peptide coupling agent.

Step D: This step involves the removal of t-Boc group with trifluoroacetic acid (TFA) followed by purification of the product by high-pressure liquid chromatography (HPLC).

The present invention further includes pharmaceutical compositions comprising a pharmaceutically effective amount of one or more of the above-described compounds as active ingredient. Pharmaceutical compositions according to the invention are suitable for enteral, such as oral or rectal, and parenteral administration to mammals, including man, for the treatment of proliferative diseases, including tumors, especially cancerous tumors, and other cancers alone or in combination with one or more pharmaceutically acceptable carriers.

The inventive compounds are useful for the manufacture of pharmaceutical compositions having an effective amount the compound in conjunction or admixture with excipients or carriers suitable for either enteral or parenteral application. Examples include tablets and gelatin capsules comprising the active ingredient together with (a) diluents; (b) lubricants, (c) binders (tablets); if desired, (d) disintegrants; and/or (e) absorbents, colorants, flavors and sweeteners. Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, the compositions may also contain other therapeutically valuable substances. The compositions are prepared according to conventional mixing, granulating or coating methods, respectively, and contain preferably about 1 to 50% of the active ingredient.

More generally, the present invention also relates to the use of the compounds of the invention for the manufacture of a medicament, in particular for the manufacture of a medicament for the treatment of proliferative diseases.

Also contemplated is the use of the pharmaceutical compositions described hereinbefore and hereinafter for the treatment of a proliferative disease.

Suitable formulations also include formulations for parenteral administration such as aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or mult-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

The pharmaceutical composition contains a pharmaceutically effective amount of the present active agent along with other pharmaceutically acceptable exicipients, carriers, fillers, diluents and the like. The term therapeutically effective amount as used herein indicates an amount necessary to administer to a host to achieve a therapeutic result, especially an anti-tumor effect, e.g., inhibition of proliferation of malignant cancer cells, benign tumor cells or other proliferative cells.

As discussed above, the compounds of the present invention are useful for treating proliferative diseases. Thus, the present invention further relates to a method of treating a proliferative disease which comprises administering a therapeutically effective amount of a compound of the invention to a mammal, preferably a human, in need of such treatment.

A proliferative disease is mainly a tumor disease (or cancer) (and/or any metastases). The inventive compounds are particularly useful for treating a tumor which is a breast cancer, genitourinary cancer, lung cancer, gastrointestinal cancer, epidermoid cancer, melanoma, ovarian cancer, pancreas cancer, neuroblastoma, head and/or neck cancer or bladder cancer, or in a broader sense renal, brain or gastric cancer; in particular (i) a breast tumor; an epidermoid tumor, such as an epidermoid head and/or neck tumor or a mouth tumor; a lung tumor, for example a small cell or non-small cell lung tumor; a gastrointestinal tumor, for example, a colorectal tumor; or a genitourinary tumor, for example, a prostate tumor (especially a hormone-refractory prostate tumor); or (ii) a proliferative disease that is refractory to the treatment with other chemotherapeutics; or (iii) a tumor that is refractory to treatment with other chemotherapeutics due to multidrug resistance.

In a broader sense of the invention, a proliferative disease may furthermore be a hyperproliferative condition such as leukemias, hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty.

Where a tumor, a tumor disease, a carcinoma or a cancer are mentioned, also metastasis in the original organ or tissue and/or in any other location are implied alternatively or in addition, whatever the location of the tumor and/or metastasis.

The inventive compound is selectively toxic or more toxic to rapidly proliferating cells than to normal cells, particularly in human cancer cells, e.g., cancerous tumors, the compound has significant antiproliferative effects and promotes differentiation, e.g., cell cycle arrest and apoptosis.

The compounds of the present invention may be administered alone or in combination with other anticancer agents, such as compounds that inhibit tumor anglogenesis, for example, the protease inhibitors, epidermal growth factor receptor kinase inhibitors, vascular endothelial growth factor receptor kinase inhibitors and the like; cytotoxic drugs, such as antimetabolites, like purine and pyrimidine analog antimetabolites; antimitotic agents like microtubule stabilizing drugs and antmitotic alkaloids; platinum coordination complexes; anti-tumor antibiotics; alkylating agents, such as nitrogen mustards and nitrosoureas; endocrine agents, such as adrenocorticosteroids, androgens, anti-androgens, estrogens, anti-estrogens, aromatase inhibitors, gonadotropin-releasing hormone agonists and somatostatin analogues and compounds that target an enzyme or receptor that is overexpressed and/or otherwise involved a specific metabolic pathway that is upregulated in the tumor cell, for example ATP and GTP phosphodiesterase inhibitors, histone deacetylase inhibitors, protein kinase inhibitors, such as serine, threonine and tyrosine kinase inhibitors, for example, Abelson protein tryosine kinase and the various growth factors, their receptors and kinase inhibitors therefore, such as, epidermal growth factor receptor kinase inhibitors, vascular endothelial growth factor receptor kinase inhibitors, fibroblast growth factor inhibitors, insulin-like growth factor receptor inhibitors and platelet-derived growth factor receptor kinase inhibitors and the like; methionine aminopeptidase inhibitors, proteasome inhibitors, and cyclooxygenase inhibitors, for example, cyclooxygenase-1 or -2 inhibitors.

The present invention further relates to a method of promoting apoptosis in rapidly proliferating cells, which comprises contacting the rapidly proliferating cells with an effective apoptosis promoting amount of a non-naturally-occurring tripeptide compound that binds to the Smac binding site of XIAP protein. Preferably, the non-naturally-occurring tripeptide compound a compound of present formula I or II.

The following examples are intended to illustrate, but not further limit, the invention.

### EXAMPLE 1

### L-(N-methyl)Ala-L-Val-(2S,4S)-4-(2-Cyclohexylacetylamino)-2-phenethylcarbamoylpyrrolidine

The title compound (Formula 1) is prepared according to the procedure set forth in Scheme 2.

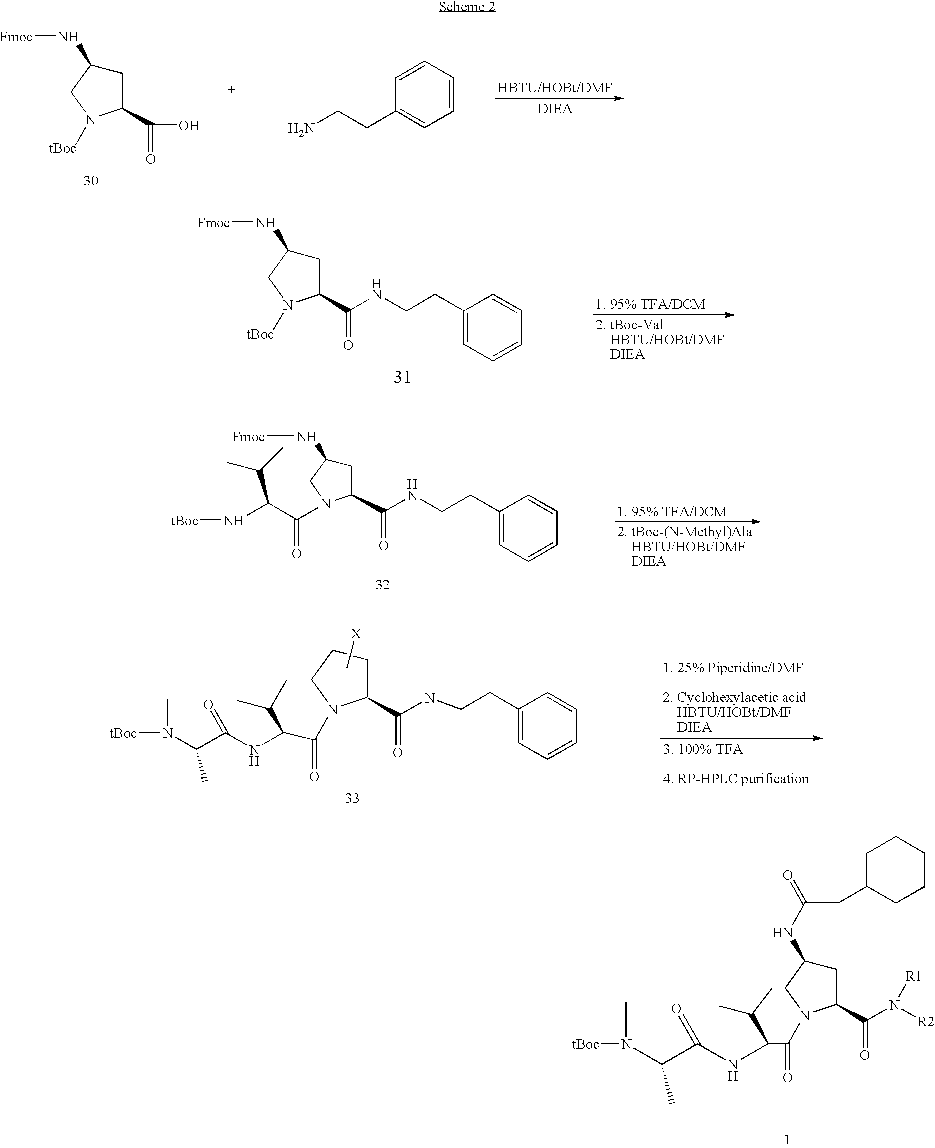

### I. Preparation of 1-tBoc(2S,4S)-4-(9H-Fluoren-9-ylmethoxycarbonylamino)-2-phenethyl-carbamoylpyrrolidine, 31

A 250 mL round-bottom flask is charged with compound 23 (3.0 9, 6.43 mmol)(see Example 1), phenethylamine (0.88 g, 7 mmol), and DIEA (30 mL). To this mixture, a 0.45 mM solution of HBTU/HOBt in DMF (15.5 mL, 7 mmol) is added and the solution stirred at room temperature overnight. The reaction mixture is diluted with EtOAc and washed well with water (2×), 10% citric acid (2×), water, brine, and dried over anhydrous MgSO₄. The EtOAc solution is concentrated in vacuum and the product purified by flash chromatography to provide 2.1 g of the title compound. Retention Time: 8.48 min (RP-HPLC, C18, 10-90%) acetonitrile/0.1% TFA gradient, 10 min); MS: ESI 555.97 (M+H)⁺.

### II. Preparation of tBoc-L-Val-(2S,4S)-4-(9H-Fluoren-9-ylmethoxycarbonylamino)-2-phenethylcarbamoylpyrrolidine, 32

A 95% solution of Trifluoroacetic acid (TFA) in methylene chloride (15 mL) was added to the compound prepared in Example 2 (2.1 g, 3.78 mM) in a 50 mL round bottom flask at rt and the solution was stirred for 1 h. The solution was concentrated in vacuume to provide a dark yellow oil. RT: 6.38 min (RP-HPLC, C18, 10-90%) acetonitrile/0.1% TFA gradient, 10 min); MS: ESI 465.3 (M+H)⁺. The crude product is combined first with DIEA (10 mL) and then tBoc-L-Val (0.8 g, 3.7 mmol) and DMF (20 mL) is added. A 0.45 mM solution of HBTU/HOBt in DMF (10 mL) is added to the reaction mixture at room temperature and the reaction mixture is stirred overnight. The reaction mixture is concentrated on a rotory evaporator and then diluted with EtOAc (150 mL) and washed well with water (2×150 mL), 10% citric acid (2×150 mL), water, brine, and dried over anhydrous MgSO₄. The EtOAc solution is concentrated in vacuum to provide 2.41 g of the title compound. Retention Time: 8.78 min (RP-HPLC, C18, 10-90%) acetonitrile/0.1% TFA gradient, 10 min); MS: ESI 784.2 (M+DIEA+H)⁺.

### III. Preparation of tBoc-L-(N-methyl)Ala-L-Val-(2S,4S)-4-(9H-Fluoren-9-ylmethoxy-carbonylamino)-2-phenethylcarbamoylpyrrolidine, 33

A 95% solution of Trifluoroacetic acid (TFA) in methylene chloride (15 mL) is added to the compound prepared in Example 3 (2.40 g) in a 50 mL round bottom flask at room temperature and the solution is stirred for 1 h. The solution is concentrated in vacuum to provide a dark yellow oil. RT: 6.62 min (RP-HPLC, C18, 10-90%) acetonitrile/0.1% TFA gradient, 10 min); MS: ESI 555.3 (M+H)⁺. The crude product is combined first with DIEA (10 mL) and then tBoc-L-(N-Me)Ala (0.8 g, 3.7 mmol) and DMF (20 mL) are added to it. A 0.45 mM solution of HBTU/HOBt in DMF (10 mL) is added to the reaction mixture at room temperature and the reaction mixture is stirred overnight. The reaction mixture is concentrated on a rotory evaporator and then diluted with EtOAc (150 mL) and washed well with water (2×150 mL), 10% citric acid (2×150 mL), water, brine, and dried over anhydrous MgSO₄. The EtOAc solution is concentrated in vacuum to provide 2.93 g of the title compound. RT: 8.80 min (RP-HPLC, C18, 10-90%) acetonitrile/0.1% TFA gradient, 10 min); MS: ESI 740.4 (M+H)⁺.

### IV. Synthesis of L-(N-methyl)Ala-L-Val-(2S,4S)-4-(2-Cyclohexylacetylamino)-2-phenethylcarbamoylpyrrolidine, 1

In a 50 mL round-bottom flask, crude compound 33 (˜2.8 g) is treated with 20 mL solution of 25% piperidine/DMF for 30 min. The mixture is concentrated on a rotory evaporator and ether was added to it. The resulting solid is filtered out and the ether layer is concentrated to provide 2.10 g of a yellow oil which is purified by RP-HPLC (C18, 10-90%) acetonitrile/0.1% TFA gradient, 30 min). Clean fractions were pooled to provide de-Fmoc product (0.97 g). RT: 5.40 min (RP-HPLC, C18, 10-90% acetonitrile/0.1% TFA gradient, 10 min); MS: ESI 518.3 (M+H)⁺. The de-Fmoc compound (0.445 g, 0.85 mmol), cyclohexylacetic acid (0.125 g, o.86 mmol) and DIEA (1.0 mL) are dissolved in 2 mL DMF. A 0.45 mM solution of HBTU/HOBt in DMF (3.0 mL) is added to the reaction mixture at room temperature and the reaction mixture is stirred overnight. The reaction mixture is concentrated on a rotory evaporator and then diluted with EtOAc (50 mL) and washed well with water (2×50 mL), 10% citric acid (2×50 mL), water, brine, and dried over anhydrous MgSO₄. The EtOAc solution is concentrated in vacuum to provide 0.53 g of a fluffy white solid. Retention Time: 8.10 min (RP-HPLC, C18, 10-90%) acetonitrile/0.1% TFA gradient, 10 min); MS: ESI no (M+H)⁺ observed. The white solid was subjected to TFA (100%, 10 mL) in a 50 mL round bottom flask at room temperature and the solution stirred for 1 h. The solution is concentrated in vacuum to provide a dark yellow oil (0.42 g). This crude product is purified by RP-HPLC (C18, 10-90%) acetonitrile/0.1% TFA gradient, 30 min). Clean fractions are pooled to provide compound 1, the title compound. Retention Time: 5.66 min (RP-HPLC, C18, 10-90%) acetonitrile/0.1% TFA gradient, 10 min); MS: ESI 542.4 (M+H)⁺.

### EXAMPLES 1-29

The following compounds are prepared by methods analagous to those described herein utilizing analogous starting materials:

| Compound Structure | Example Number |
|---|---|
| 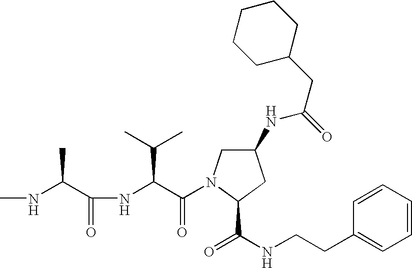 | Example 1 MS ESI 542.4 (M + H)⁺ |
| 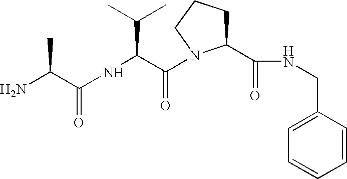 | Example 2 MS ESI 375.4 (M + H)⁺ |
| 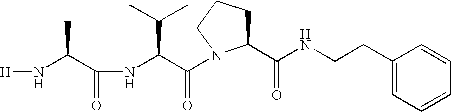 | Example 3 MS ESI 389.4 (M + H)⁺ |
| 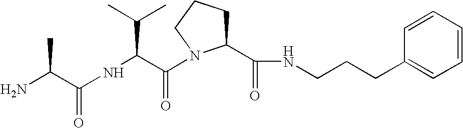 | Example 4 MS ESI 403.4 (M + H)⁺ |
| 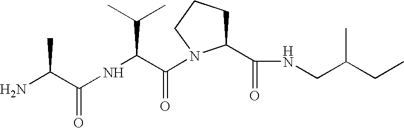 | Example 5 MS ESI 355.4 (M + H)⁺ |
| 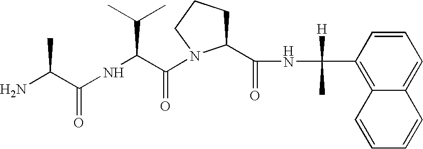 | Example 6 MS ESI 439.4 (M + H)⁺ |
| 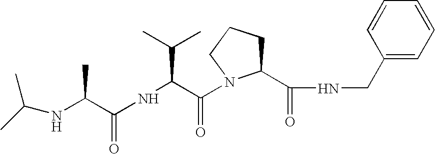 | Example 7 MS ESI 417.6 (M + H)⁺ |
| 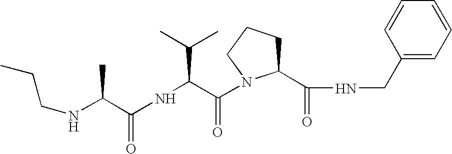 | Example 8 MS ESI 417.6 (M + H)⁺ |
| 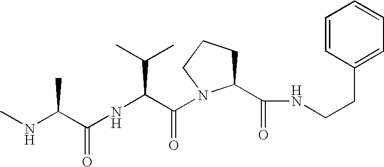 | Example 9 MS ESI 403.2 (M + H)⁺ |
| 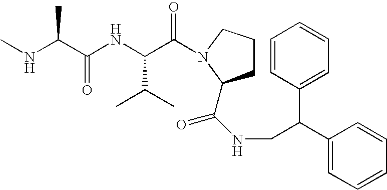 | Example 10 MS ESI 479.3 (M + H)⁺ |
| 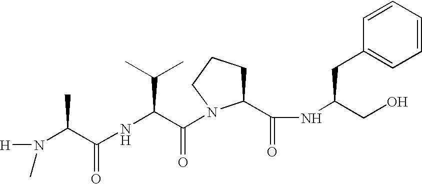 | Example 11 MS ESI 433.1 (M + H)⁺ |
| 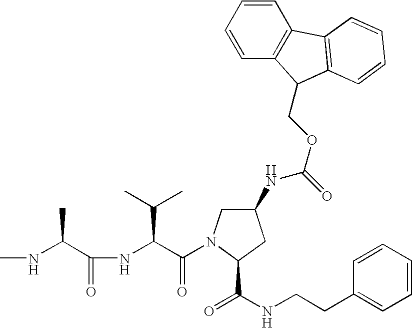 | Example 12 MS ESI 640.2 (M + H)⁺ |
| 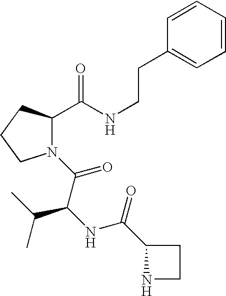 | Example 13 MS ESI 401.6 (M + H)⁺ |
| 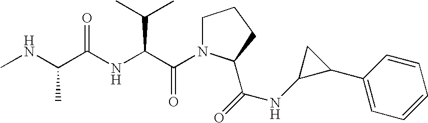 | Example 14 MS ESI 415.5 (M + H)⁺ |
| 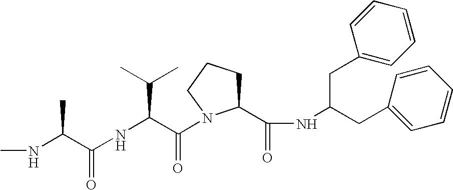 | Example 15 MS ESI 478.4 (M + H)⁺ |
| 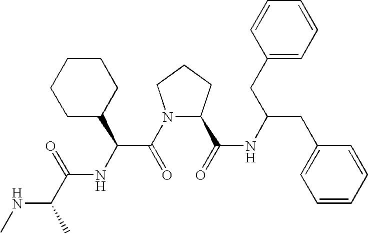 | Example 16 MS ESI 533.6 (M + H)⁺ |
| 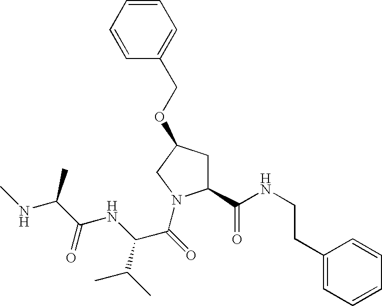 | Example 17 MS ESI 509.5 (M + H)⁺ |
| 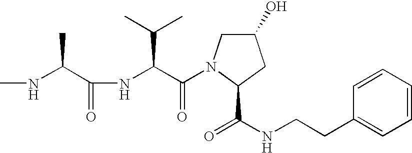 | Example 18 MS ESI 419.3 (M + H)⁺ |
| 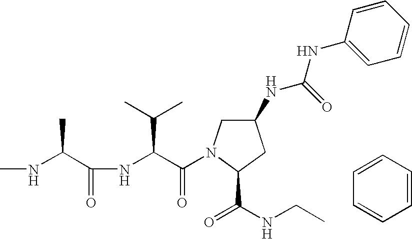 | Example 19 MS ESI 537.2 (M + H)⁺ |
| 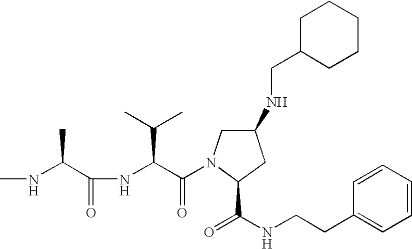 | Example 20 MS ESI 514.3 (M + H)⁺ |
| 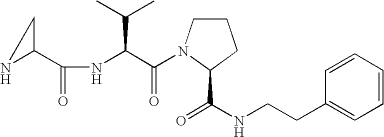 | Example 21 MS ESI 387.3 (M + H)⁺ |
| 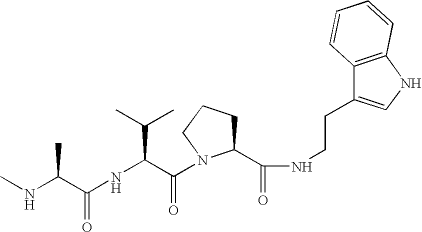 | Example 22 MS ESI 442.7 (M + H)⁺ |
| 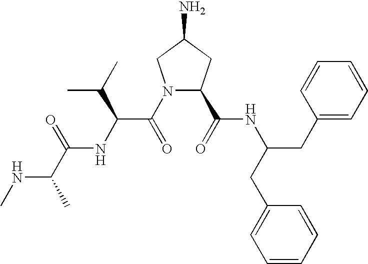 | Example 23 MS ESI 508.7 (M + H)⁺ |
| 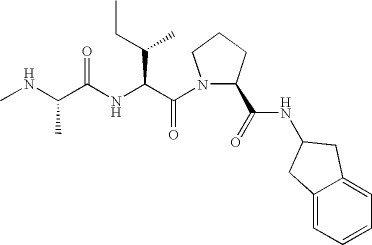 | Example 24 MS ESI 429.4 (M + H)⁺ |
| 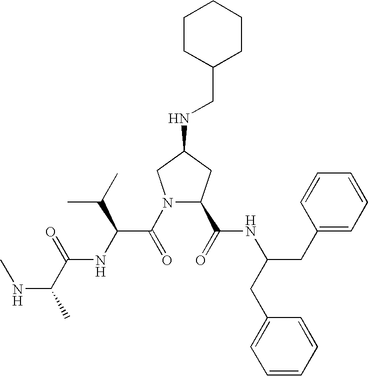 | Example 25 MS ESI 604.7 (M + H)⁺ |
| 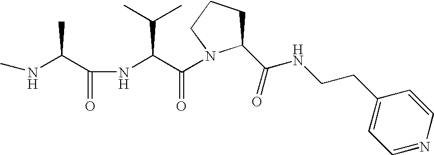 | Example 26 MS ESI 404.3 (M + H)⁺ |
| 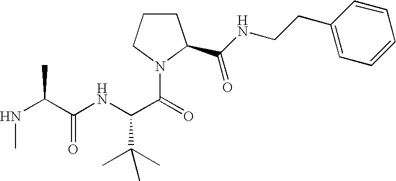 | Example 27 MS ESI 417.6 (M + H)⁺ |
| 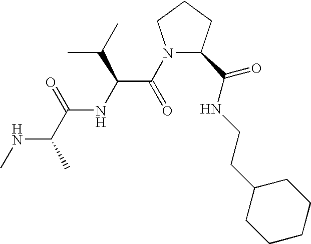 | Example 28 MS ESI 409.6 (M + H)⁺ |
| 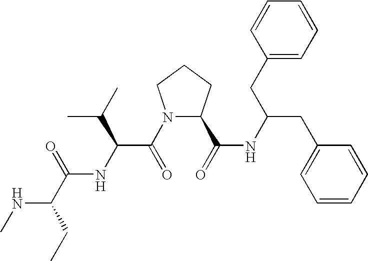 | Example 29 MS ESI 507.6 (M + H)⁺ |

In order to measure the ability of the inventive compounds to bind the BIR3 peptide binding pocket, a solution phase assay on the FMAT technology platform is utilized. Biotinylated Smac 7-mer peptide (AVPIAQK, lysine ε-amino group is biotinylated) is immobilized on streptavidin coated beads. GST-BIR3 fusion protein is precipitated with FMAT beads and is detected using fluorescent tagged anti-GST antibodies. Importantly, non-biotinylated Smac peptide is highly effective at competing GST-BIR3 off the FMAT beads (FIG. 2). The IC₅₀ for non-biotinylated Smac is 400 nM. The IC₅₀values of compounds listed in Table 1 in the described FMAT assay ranged from 0.045-10 μM.

1-14. (canceled)

15. A compound of the formula (I) 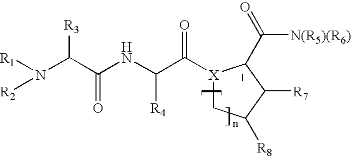
wherein
R₁ is H;
R₂ is H, C₁-C₄alkyl which is unsubstituted or substituted by one or more substituents selected from halogen, —OH, —SH, —OCH₃, —SCH₃, —CN, —SCN and nitro;
R₃ is H, —CF₃, —C₂F₅, —CH₂-Z or R₂ and R₃ together form with the nitrogen form a C₃-C₆heteroaliphatic ring;
Z is H, —OH, F, Cl, —CH₃; —CF₃, —CH₂Cl, —CH₂F or —CH₂OH;
R₄ is C₁-C₁₆ straight chain alkyl, C₃-C₁₀ branched chain alkyl, —(CH₂)₀₋₆—C₃-C₇-cycloalkyl, —(CH₂)₁₋₆-Z₁, —(CH₂)₀₋₆-phenyl, and —(CH₂)₀₋₆-het, wherein the alkyl, cycloalkyl and phenyl substituents are unsubstituted or substituted;
Z₁ is —N(R₉)—C(O)—C₁-C₁₀alkyl, —N(R₉)—C(O)—(CH₂)₁₋₆—C₃-C₇-cycloalkyl, —N(R₉)—C(O)—(CH₂)₀₋₆-phenyl, —N(R₉)—C(O)—(CH₂)₁₋₆-het, —C(O)—N(R₁₀)(R₁₁), —C(O)—O—C₁-C₁₀alkyl, —C(O)—O—(CH₂)₁₋₆—C₃-C₇-cycloalkyl, 'C(O)—O—(CH₂)₀₋₆-phenyl, —C(O)—O—(CH₂)₁₋₆-het, —O—C(O)—C₁-C₁₀alkyl, —O—C(O)—(CH₂)₁₋₆—C₃-C₇-cycloalkyl, —O—C(O)—(CH₂)₀₋₆-phenyl, —O—C(O)—(CH₂)₁₋₆-het, wherein the alkyl, cycloalkyl and phenyl substituents are unsubstituted or substituted;
het is a 5-7 membered heterocyclic ring containing 1, 2 or 3 heteroatoms selected from N, O and S, or an 8-12 membered fused ring system including at least one 5-7 membered heterocyclic ring containing 1, 2 or 3 heteroatoms selected from N, O, and S, which heterocyclic ring or fused ring system is unsubstituted or substituted on a carbon atom by halogen, hydroxy, C₁-C₄alkyl, C₁-C₄ alkoxy, nitro, —O—C(O)—C₁-C₄alkyl or —C(O)—O—C₁-C₄-alkyl or on a nitrogen by C₁-C₄ alkyl, —O—C(O)—C₁-C₄alkyl or —C(O)—O—C₁-C₄-alkyl;
R₉ is H, —CH₃, —CF₃, —CH₂OH or CH₂Cl;
R₁₀ and R₁₁ are each independently H, C₁-C₄alkyl, C₃-C₇-cycloalkyl, —(CH₂)₁₋₆—C₃-C₇-cycloalkyl, —(CH₂)₀₋₆-phenyl, wherein the alkyl, cycloalkyl and phenyl substituents are unsubstituted or substituted, or R₁₀ and R₁₁ together with the nitrogen are het;
X is CH or N;
R₅ is H, C₁-C₁₀-alkyl, C₃-C₇-cycloalkyl, —(CH₂)₁₋₆—C₃-C₇-cycloalkyl, —C₁-C₁₀-alkyl-aryl, —(CH₂)₀₋₆—C₃-C₇-cycloalkyl-(CH₂)₀₋₆-phenyl, —(CH₂)₀₋₄CH—((CH₂)₁₋₄-phenyl)₂, —(CH₂)₀₋₆CH(phenyl)₂, —C(O)—C₁-C₁₀alkyl, —C(O)—(CH₂)₁₋₆—C₃-C₇-cycloalkyl, —C(O)—(CH₂)₀₋₆-phenyl, —(CH₂)₁₋₆-het, —C(O)—(CH₂)₁₋₆-het, wherein the alkyl, cycloalkyl, phenyl and aryl substituents are unsubstituted or substituted;
R₆ is H, methyl, ethyl, —CF₃, —CH₂OH or —CH₂Cl; or
R₅ and R₆ together with the nitrogen are het;
R₇ and R₈ are cis relative to the acyl substituent at the one position of the ring and are each independently H, —C₁-C₁₀ alkyl, —OH, —O—C₁-C₁₀-alkyl, —(CH₂)₀₋₆—C₃-C₇-cycloalkyl, —O—(CH₂)₀₋₆-aryl, phenyl, —(CH₂)₁₋₆-het, —O—(CH₂)₁₋₆-het, —N(R₁₂)(R₁₃), —S—R₁₂, —S(O)—R₁₂, —S(O)₂—R₁₂, —S(O)₂—NR₁₂R₁₃ wherein the alkyl, cycloalkyl and aryl substituents are unsubstituted or substituted;
R₁₂ and R₁₃ are independently H, C₁-C₁₀ alkyl, —(CH₂)₀₋₆—C₃-C₇-cycloalkyl, —(CH₂)₀₋₆—(CH)₀₋₁(aryl)₁₋₂, —C(O)—C₁-C₁₀alkyl, —C(O)—(CH₂)₁₋₆—C₃-C₇-cycloalkyl, —C(O)—O—(CH₂)₀₋₆-aryl, —C(O)—(CH₂)₀₋₆—O-fluorenyl, —C(O)—NH—(CH₂)₀₋₆-aryl, —C(O)—(CH₂)₀₋₆-aryl, —C(O)—(CH₂)₁₋₆-het, wherein the alkyl, cycloalkyl and aryl substituents are unsubstituted or substituted; or a substituent that facilitates transport of the molecule across a cell membrane, or R₁₂ and R₁₃ together with the nitrogen are het;
aryl is phenyl or naphthyl which is unsubstituted or substituted;
n is 0, 1 or 2;
and wherein
substituted alkyl substituents are substituted by one or more substituents selected from a double bond, halogen, OH, —O—C₁-C₆alkyl, —S—C₁-C₆alkyl and —CF₃;
substituted cycloalkyl substituents are substituted by one or more substituents selected from a double bond, C₁-C₆alkyl, halogen, OH, —O—C₁-C₆alkyl, —S—C₁-C₆alkyl and —CF₃; and
substituted phenyl or aryl are substituted by one or more substituents selected from halogen, hydroxy, C₁-C₄ alkyl, C₁-C₄ alkoxy, nitro, —CN, —O—C(O)—C₁-C₄alkyl and —C(O)—O—C₁-C₄-alkyl, or a pharmaceutically acceptable salt thereof.

16. A compound of claim 15 wherein R₂ is H or methyl and R₃ is methyl.

17. A compound of claim 15 wherein n is 1.

18. A compound of claim 15 having the stereochemistry indicated in formula II

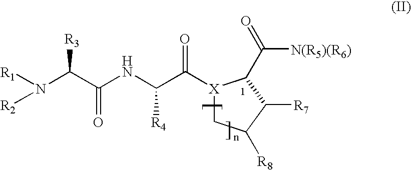

19. A compound of claim 18 wherein R₂ is H or methyl and R₃ is methyl.

20. A compound of claim 18 wherein n is 1.

21. A pharmaceutical composition which comprises a pharmaceutically acceptable carrier and a therapeutically effective amount of a compound of formula I according to claim 15.

22. A pharmaceutical composition which comprises a pharmaceutically acceptable carrier and a therapeutically effective amount of a compound of formula II according to claim 18.

23. A pharmaceutical composition according to claim 21 for treating a proliferative disease.

24. A pharmaceutical composition according to claim 22 for treating a proliferative disease.

25. A method of treating a proliferative disease which comprises administering a therapeutically effective amount of a compound of formula I according to claim 15 to a mammal in need of such treatment.

26. A method of treating a proliferative disease which comprises administering a therapeutically effective amount of a compound of formula II according to claim 18 to a mammal in need of such treatment.

27. A method of claim 25 wherein the mammal is a human.

28. A method of claim 26 wherein the mammal is a human.
